(19) United States
(12) Patent Application Publication
(10) Pub. No.: US 2016/0080696 A1
(43) Pub. Date: Mar. 17, 2016

(54) Contact Area Diffusion Factor for Quantifying Fat Contents of Liquid

(71) Applicant: Sanghyun Lee, Pohang (KR)

(72) Inventor: Sanghyun Lee, Pohang (KR)

(21) Appl. No.: 14/947,980

(22) Filed: Nov. 20, 2015

Related U.S. Application Data
(62) Division of application No. 14/106,992, filed on Dec. 16, 2013.

(30) Foreign Application Priority Data
Dec. 17, 2012 (KR) 10-2012-0147029

(51) Int. Cl. H04N 7/18; G06T 7/00; H04N 5/232; G01N 33/487

(52) U.S. Cl. CPC H04N 7/18; G01N 33/487; G06T 7/0012; H04N 5/23296

(57) ABSTRACT

A method for determining the presence of fats in the body fluid by photographing a body fluid droplet and calculating the change of the contact area of the body fluid droplet and the contact area diffusion factor. In addition, fats may be easily detected by using a simple filming equipment where it is required to detect fats such as liposuctions, various orthopedic operations, obesity managements, etc.

### CROSS-REFERENCE TO RELATED APPLICATIONS

This application is a Divisional of U.S. patent application Ser. No. 14/106,992, filed Dec. 16, 2013, which claims priority to Korea Patent Application No. 10-2012-0147029, filed Dec. 17, 2012, the disclosures of which are incorporated herein by reference.

### FIELD OF THE INVENTION

The present invention relates to a method for measuring the fat contents of liquid from the CADF value of the liquid droplet.

### BACKGROUND OF THE INVENTION

Liposuction is a plastic operation (surgery) which is the most frequently performed among 21 categories of plastic surgery, over the world including Korea. According to the 2009 report of the ISAPS, over 1.6 millions of liposuctions were performed worldwide.

According the above-mentioned report, sixty five thousand liposuctions were performed in Korea (FIG. 1). It is expected that much more liposuctions were performed, considering liposuctions which were not included in the report.

Although liposuction is the most frequently-performed operation among plastic operations, risks that occur during liposuction are not well-known to the public. This is because, on the one hand, mortality accidents due to side effects of liposuction is much fewer than the number of liposuction operations and, on the other hand, most of the mortality accidents are not reported by the press. This is also because FES (fat embolism syndrome) which would be occurred by introduction of fat into blood vessels during liposuction, is known not as a risk of liposuction, but as a medical accident.

Although all the mechanisms of FES has not yet revealed, it is well-known that the most important cause of FES is that fats passing into damaged blood vessels during liposuction may seriously deteriorate the function of the lung or penetrate into other organs, thereby causing death. Presently, it is known that the possibility of inducing FES is proportional to the amount of fats passed into blood stream during liposuction and, accordingly, the mortality risk increases.

Therefore, in order to prevent FES during liposuction, it is necessary to measure the amount of fats passed into blood vessels before, during and after an operation. However, there are no diagnostic apparatuses relating to measuring the amount of fats passed into blood vessels.

The present inventor has completed the present invention by confirming that the fat content of liquid may be measured from the CADF (contact area diffusion factor) values of a liquid droplet.

### SUMMARY OF THE INVENTION

The primary object of the present invention is to provide a method for measuring fat contents in a liquid based on a contact area diffusion factor, comprising: placing liquid on a water-repellant surface as a droplet (s100); obtaining a magnified image from an image of said droplet (s200); obtaining a contact diameter (d₀) from said magnified image (s300); obtaining a contact diameter, d₍ₜ₎, after the lapse of predetermined time (t) from said magnified image (s400); and calculating a contact area diffusion factor (CADF) by substituting said d₍ₜ₎ and d₀ for the following equation 1 (s500);

$$C A D F = \frac{{d}_{( t )}^{2} - {d}_{0}^{2}}{{d}_{0}^{2}} \times 100 \qquad ( Equation 1 )$$

where d₍ₜ₎ is the contact diameter of said droplet after the lapse of time, t, and d₀ is the initial contact diameter.

Another object of the present invention is to provide a method for measuring fat contents in a liquid based on a contact area diffusion factor, comprising: placing liquid on a water-repellant surface as a droplet (s1100); obtaining a magnified image from an image of said droplet (s1200); obtaining a contact area (A(0)) between said repellant surface and said droplet from said magnified image (s1300); and obtaining a contact area, A(t), between said repellant surface and said droplet after the lapse of predetermined time (t) from said magnified image (s1400), wherein said fat is determined to be present in said body fluid according to whether the value of A(t)−A(0) is positive or negative.

Yet another object of the present invention is to provide a method for measuring fat contents in a liquid based on a contact area diffusion factor, comprising: placing liquid on a water-repellant surface as a droplet (s2100); obtaining a magnified image from an image of said droplet (s2200); obtaining a contact diameter (dₒ) from said magnified image (s2300); and obtaining a contact diameter, d₍ₜ₎, after the lapse of predetermined time (t) from said magnified image (s2400), wherein said fat is determined to be present in said body fluid according to whether the value of d₍ₜ₎−d₀ is positive or negative.

In other words, the object of the present invention is to provide a method for determining the presence of fats in the body fluid by photographing a body fluid droplet and calculating the change of the contact area of the body fluid droplet and the contact area diffusion factor.

In addition, the present invention is to provide a method for determining the presence of fats by using a simple filming equipment where it is required to detect fats such as liposuctions, various orthopedic operations, obesity managements, etc.

### DETAILED DESCRIPTION OF THE INVENTION

The primary object of the present invention can be accomplished by providing a method for measuring fat contents in a liquid based on a contact area diffusion factor, comprising:

placing liquid on a water-repellant surface as a droplet (s100);

obtaining a magnified image from an image of said droplet (s200);

obtaining a contact diameter (dₒ) from said magnified image (s300);

obtaining a contact diameter, d₍ₜ₎, after the lapse of predetermined time (t) from said magnified image (s400); and

calculating a contact area diffusion factor (CADF) by substituting said d₍ₜ₎ and d₀ for the following equation 1 (s500);

$$C A D F = \frac{{d}_{( t )}^{2} - {d}_{0}^{2}}{{d}_{0}^{2}} \times 100 \qquad ( Equation 1 )$$

where d₍ₜ₎ is the contact diameter of said droplet after the lapse of time, t, and d₀ is the initial contact diameter.

As used herein, term “fat” shall include both solid fats (m.p. above 20° C.) and liquid fats (i.e., oils) unless otherwise specifically indicated. Fats and oils are generally recognized to be fatty acid triglycerides which are either naturally occurring in vegetable and animal fats and oils, but also include rearranged or randomized fats and oils and interesterified fats and oils.

The term “fatty acid”, as used herein, refers to saturated and or unsaturated (including mono-, di- and poly-unsaturated) straight chain carboxylic acids having from 12 to 24 carbon atoms.

As used herein, the term “trans fat” refers to a type of unsaturated fat containing trans fatty acids. Trans fats increase the LDL cholesterol, while also lowering the HDL cholesterol in blood. The term “trans fatty acid”, as used herein, refers to a fatty acid that is commonly produced by the partial hydrogenation of the unsaturated fatty acid vegetable oils. The term “trans” refers to the opposed positioning of hydrogen atoms when unsaturated fats are partially hydrogenated.

As used herein, the term “contact angle” refers to an angle that formed between the surface of a solid and the line tangent to the droplet radius from the point of contact with the solid.

As used herein, the term “body fluid” refers to human or animal serum, plasma, sweat, urine and the like.

The liquid may be a body fluid and the body fluid may be selected from serum, plasma, sweat or urine.

Another object of the present invention can be accomplished by providing a method for measuring fat contents in a liquid based on a contact area diffusion factor, comprising:

placing liquid on a water-repellant surface as a droplet (s1100);

obtaining a magnified image from an image of said droplet (s1200);

obtaining a contact area (A(0)) between said repellant surface and said droplet from said magnified image (s1300); and

obtaining a contact area, A(t), between said repellant surface and said droplet after the lapse of predetermined time (t) from said magnified image (s1400),

wherein said fat is determined to be present in said body fluid according to whether the value of A(t)−A(0) is positive or negative.

The liquid may be a body fluid and the body fluid may be selected from serum, plasma, sweat or urine.

Yet another object of the present invention can be accomplished by providing a method for measuring fat contents in a liquid based on a contact area diffusion factor, comprising:

placing liquid on a water-repellant surface as a droplet (s2100);

obtaining a magnified image from an image of said droplet (s2200);

obtaining a contact diameter (d₀) from said magnified image (s2300); and

obtaining a contact diameter, d₍ₜ₎, after the lapse of predetermined time (t) from said magnified image (s2400),

wherein said fat is determined to be present in said body fluid according to whether the value of d₍ₜ₎−d₀ is positive or negative.

The liquid may be a body fluid and the body fluid may be selected from serum, plasma, sweat or urine.

### Advantageous Effects

The present invention provides the following technical effects.

It is possible to determine whether fats are present and how much the fats exist in a body fluid by calculating the change of the contact area and the contact area diffusion factor (CADF) from an image of a body fluid droplet.

In addition, fat embolism syndrome may be prevented since the presence of fats can be discovered early through the present invention.

Accordingly, fat contents may be measured during liposuction, and fat embolism syndrome may be prevented through the present invention. Moreover, the present invention may be used to detect fats when various orthopedic operations and chemical analyses are performed. Further, the present invention may be utilized to monitor an individual's fat metabolism to manage obesity, diet, health care, etc.

### BRIEF DESCRIPTION OF THE DRAWINGS

FIG. 1 shows statistical data of liposuctions performed worldwide in 2009 by the ISAPA.

FIG. 2 illustrates three surface tensions acting along the contact line between a droplet and a surface of a bottom.

FIG. 3 is a graph that shows the CADF values of the urine containing fats after liposuction and the urine containing no fats before liposuction.

FIG. 4 is a graph that shows the CADF values of the urine samples collected each 0.5 days after liposuction.

FIG. 5 is a graph that shows the CADF values of the urine samples collected each 0.5 days after liposuction.

FIG. 6 is a graph showing the change of the CADF with the change of the concentrations of the urine samples collected after liposuction.

FIG. 7 shows a table that lists the types and concentrations of the free fatty acids, which were measured by gas chromatography (GC), and the free fatty acid were detected by using the CADF values.

FIG. 8 shows the total fatty acids concentration (C_{CADF}) and the total fatty acid concentration obtained by adding all the fatty acid concentrations measured by GC.

FIG. 9 is a graph showing the four important fatty acid concentrations and the total fatty acid concentration calculated from the CADF.

FIG. 10 is a graph showing the change of the CADF with diluting the blood plasma samples.

FIG. 11 is a graph showing the LDL (low density lipoprotein) values obtained from the hyperlipidemia test results for the blood samples and the CADF values measured for the same blood samples.

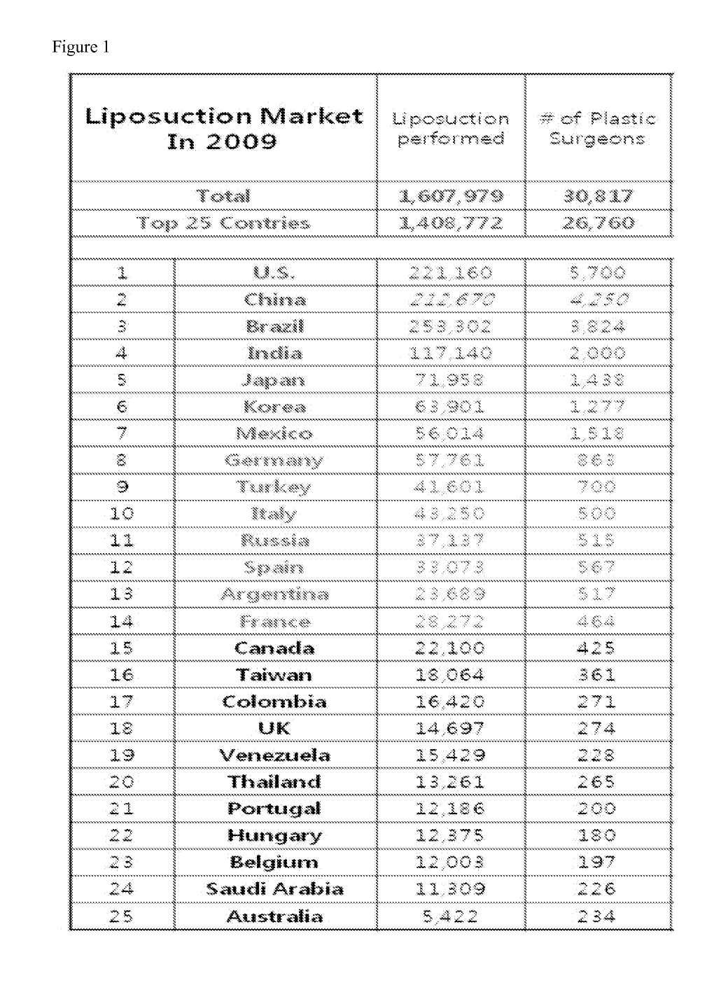

### BEST MODE FOR CARRYING OUT THE INVENTION

Hereinbelow, the present invention will be described in greater detail with reference to the following embodiments and drawings. However, the embodiments and drawings are given only for illustration of the present invention and not to be limiting the present invention.

As described in the above, it is known that, as the amount of fats passed into blood stream is larger, the possibility of onset of FES is higher and, accordingly, the mortality risk is higher.

Therefore, in order to prevent FES during liposuction, it is essential to measure quantitatively the amount of the fats passed into blood stream, before, during and after liposuction and, however, there is no diagnostic apparatus in this regard.

The present invention provides a method for determining whether a body fluid contains fats from images of the body fluid droplet.

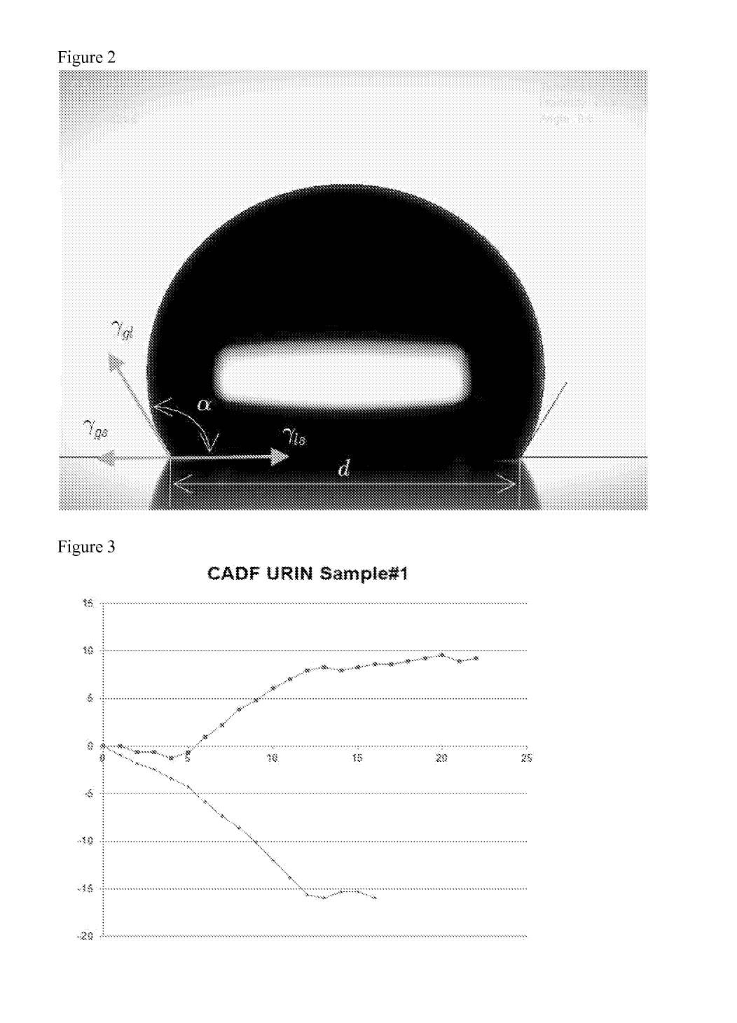

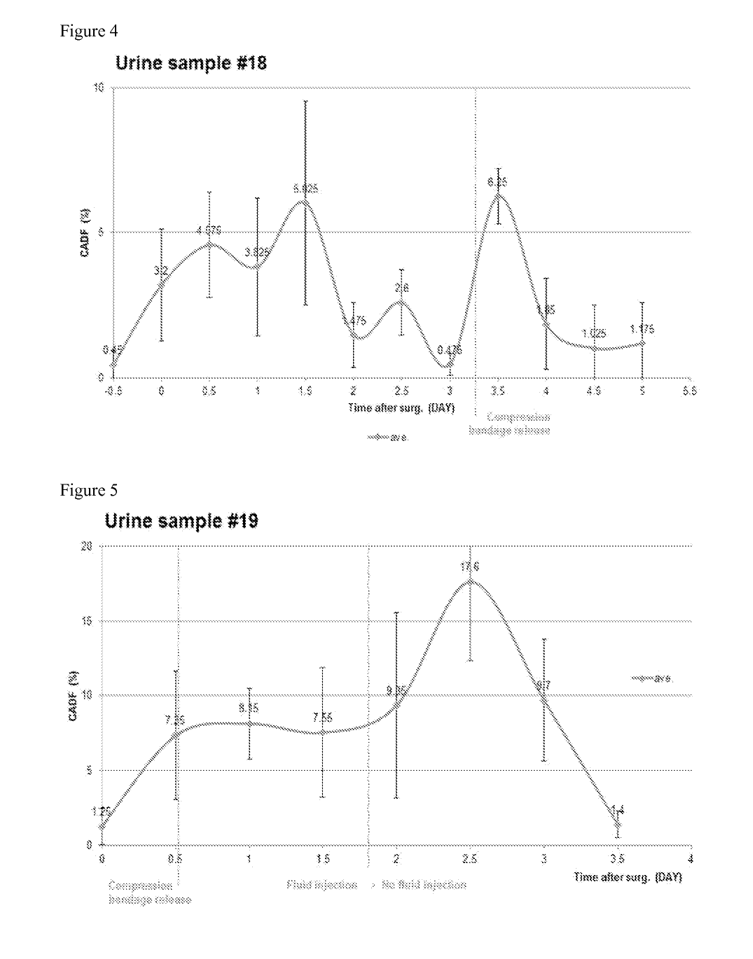

FIG. 2 illustrates three surface tensions acting on the contact line between a droplet and a surface of a bottom, FIG. 3 is a graph that shows the CADF values of the urine containing fats after liposuction and the urine containing no fats before liposuction, FIG. 4 is a graph that shows the CADF values of the urine samples collected each 0.5 days after liposuction, and FIG. 5 is a graph that shows the CADF values of the urine samples collected each 0.5 days after liposuction.

With reference to FIG. 2, blood or urine samples from a patient which were pre-treated were placed, as a micro-droplet, on the specially treated surface and an image of the droplet is shown.

As shown in FIG. 2, when an image of a hydrophilic micro-droplet placed on the solid surface (this droplet is referred to as a sessile droplet) is magnified, it can be understood that a certain contact angle between the micro-droplet and the surface is maintained by surface tensions.

The micro-droplet may be body fluids such as blood, urine, etc. and any other fluid substance.

In FIG. 2, γ_{gl} is a surface tension between gas and liquid, γₗₛ is a surface tension between liquid and solid surface, and γ_{gs} is a surface tension between gas and solid surface. In addition, a is a contact angle of the micro-droplet, and d is a contact diameter between the micro-droplet and the solid surface.

It is a known technique to measure a contact diameter from an image of a liquid droplet and, thus, detailed description thereof is not provided herein.

Water is evaporated from the micro-droplet as time goes by and, simultaneously, the contact area between the micro-droplet and the solid surface changes.

Considering the changes due to evaporation of water from the micro-droplet, the contact diameter and the contact area decrease as the volume of the micro-droplet decreases.

The contact area decreases during evaporation since the force that tends to maintain the shape of the micro-droplet acts.

That is, the equilibrium between the three surface tensions (γ_{gl}: a surface tension between gas and liquid, γₗₛ: a surface tension between liquid and solid surface, and γ_{gs}: a surface tension between gas and solid surface) shown in FIG. 2 exists. However, if the contact area does not decrease when the volume of the micro-droplet decreases due to evaporation of water, the equilibrium between the three surface tensions will be broken and force will act in the direction that reduces the contact area and, accordingly, the contact area and the contact diameter (d) will decrease.

If the micro-droplet does not contain fats, the contact area does not expand (and the contact angle tends to be constant) after evaporation of water. However, if the micro-droplet contains fats, the concentration changes due to evaporation and the surface tension between the liquid and the solid surface changes due to fats attached to the solid surface after lapse of time and, therefore, the contact area and angle of the micro-droplet reduce to become flat-shaped.

$$C A D F = \frac{{d}_{( t )}^{2} - {d}_{0}^{2}}{{d}_{0}^{2}} \times 100 \qquad ( Equation 1 )$$

In the equation 1, d₍ₜ₎ is a contact diameter after lapse of time t, and d₀ is an initial contact diameter.

As used herein, these degree of change of the contact area is referred to as a contact area diffusion factor (CADF).

If a liquid droplet contains fats and is placed on a water-repellent surface, the water-repellent surface is not sufficiently wetted with water while fats such as oils strongly attach to the water-repellent surface. Therefore, fats contained in the liquid droplet attach to the water-repellent surface.

During attachment of the fats to the water-repellent surface, the surface tension between the liquid droplet and the solid surface (Δₗₛ) decreases and, thus, the contact angle decreases and the contact area increases. This contact area changes larger when the amount of the fats in the liquid droplet is larger.

When the liquid droplet does not contain fats, the contact area or the contact diameter (d) becomes smaller than the initial value; and when the liquid droplet contains fats, the contact area or the contact diameter (d) becomes larger than the initial value. The CADF represent this tendency.

As shown in Eq. 1, the CADF equals the difference between the square of the initial contact diameter (d₀²) and the contact diameter (d₍ₜ₎²) which is changing during evaporation divided by the square of the initial contact area, which is dimensionless. If the contact area decreases, the CADF is negative (−), and vice versa. The amount of fat content of the liquid droplet may be quantified according to the absolute value of the CADF.

FIG. 3 is a graph that shows the CADF values of the urine containing fats after liposuction and the urine containing no fats before liposuction. It is possible to detect quantitatively the amount of the fats contained in the liquid droplet, and it took about 20 minutes to measure the CADF. The time for the measurement may become shorter or longer, depending on the conditions of the evaporation.

In contrast with the CADF of the urine containing no fats (blue line), the CADF of the urine containing fats (red line) is positive and increasing.

In FIG. 3, the horizontal axis represents time (min) and the vertical axis represents the value of the CADF.

With reference to FIG. 3, the CADF value of urine with no fats are always measured to be negative. That is, the contact area and contact angle of the urine droplet with no fats decrease as water evaporates since the contact angle maintains despite evaporation of water.

However, as shown by the red line in FIG. 3, the CADF of the urine containing fats, e.g., a urine sample obtained from a patient with liposuction, is positive.

This indicates that the shape of the liquid droplet at time t is flatter than that of the initial liquid droplet.

Especially, it has been discovered that changes of other factors except the fat content, e.g., ion concentration or pH value, do not influence the CADF.

Therefore, the CADF is positive only when fats are contained in the liquid droplet. Thus, it can be determined from the CADF whether or not fats pass into blood streams. Moreover, it can be understood that the amount of fats in the liquid droplet corresponds to the absolute value of the CADF.

When the absolute value of the CADF of the blood collected from a patient after liposuction is large, it is possible to determine that much fats passed into blood vessels during liposuction. This may be an indication that a patient may become in a serious condition due to onset of fat embolism syndrome.

Specifically, when fats abruptly pass into blood stream, the fats are discharged through urine, sweat, tears, etc. in order to remove the fats from blood stream. Thus, FES may be diagnosed from body fluids other than blood.

FIG. 4 is a graph that shows the CADF values of the urine samples collected each 0.5 days after liposuction. The average and standard deviation were calculated from four simultaneous measurements per a sample.

The horizontal axis in FIG. 4 represents days after liposuction. The CADFs were measured at every 12 hours from 0.5 days (12 hours) before liposuction to 5 days after liposuction.

With reference to FIG. 4, most of the CADF values are positive. In addition, most of the CADF values measured one month after measuring the samples were relatively small. Therefore, it can be understood that the fat content of the urine decreased with lapse of time.

In the case of the patient in FIG. 4, the CADF value after 3.5 days is the largest for the reason that blood circulation of the patient became good and, thus, the amount of fats discharged through urine increased when compression bandages for the abdomen and the femoral, which were used for suppressing blood circulation after operation (surgery), region were removed. This indicates that medical services influence the amount of fat discharge.

It can be understood that the measurement of the CADF is sensitively influenced by various medical services (e.g., compression of the lesion, supply of infusion solution, etc.). In addition, this demonstrates that the measured value of the CADF sensitively reflects the amount of fats contained in a body fluid.

FIG. 5 is a graph that shows the CADF values of the urine samples collected each 0.5 days after liposuction, from other patient who is not the same patient as the patient in FIG. 4.

The average and standard deviation were calculated from four measurements per a sample. The patient had been hospitalized 1.5 days after surgery (liposuction) with infusion of Ringer's solution, and did not infused with Ringer's solution 2 days after surgery.

The CADF was the largest at 2.5 days since the concentration of the fat increased due to cessation of infusing Ringer's solution. As shown in FIG. 4, fats did not remain in the urine 3.5 days after liposuction.

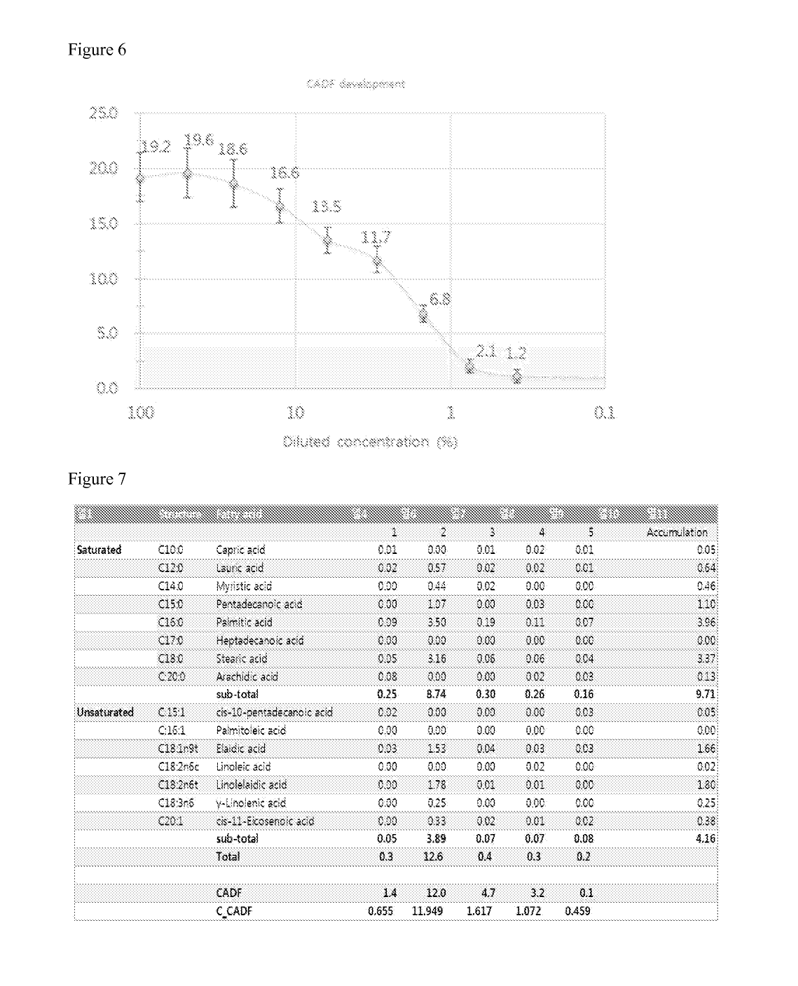

FIG. 6 is a graph showing the change of the CADF with the change of the concentration, diluting with deionized water the urine samples collected after liposuction from 100% to 0.4%. It can be understood from FIG. 6 that fats (e.g., free fatty acids, etc.) that exist in the urine samples may be quantitatively measured from the CADF. From the results of the measurements, the correlation between the CADF and the concentration (C) of the free fatty acids contained in the urine may be obtained as the following equation 2.

$$C = {e}^{\frac{CADF - 5.53}{3.65}} \qquad ( Equation 2 )$$

FIG. 7 shows a table that lists the types and concentrations of the free fatty acids, which were measured by gas chromatography (GC), and the free fatty acid were detected by using the CADF values. According to the results of the measurement, the most abundant fatty acids among the fatty acids that newly detected in the urine after liposuction were two saturated fatty acids and two unsaturated fatty acids. Saturated fatty acids and unsaturated trans fatty acids are harmful and are typical materials that cause cardiovascular diseases since these materials are solid at ambient temperature due to their high melting point and, thus, they tend to stick to the vessel wall. Considering the above, any substances that are adhesive may be easily detected from the CADF value.

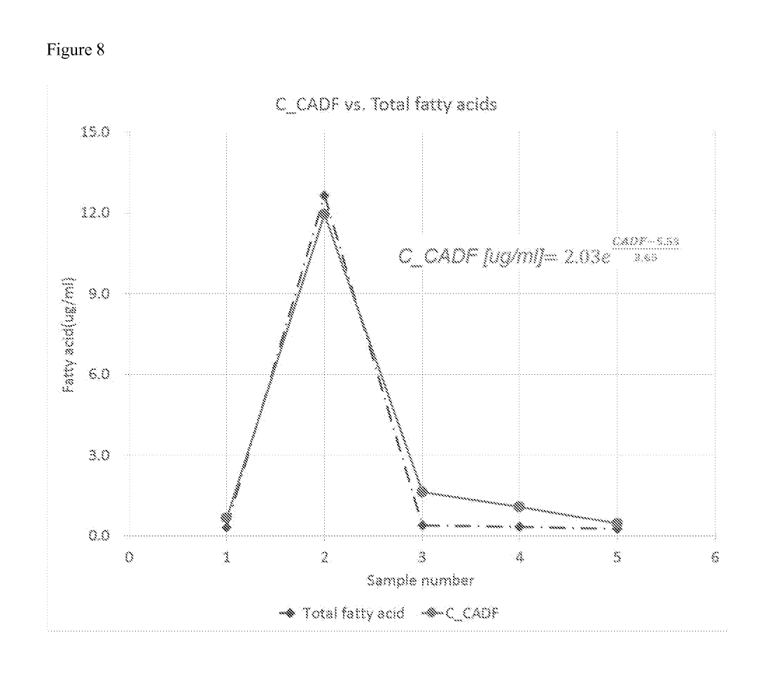

The correlation between the CADF and the amount of fatty acids contained in the urine may be obtained, based on the characteristics of the CADF and the results of analysis of fatty acids contained in the urine, in FIGS. 6 and 7. The thus obtained total fatty acids concentration (C_{CADF}) and the total fatty acid concentration obtained by adding all the fatty acid concentrations measured by GC are compared in FIG. 8. The total fatty acid concentration in the urine may be calculated from the CADF by the following equation 3.

$${C}_{C A D F} ( ug \text{/} ml ) = 2.03 {e}^{\frac{CADF - 5.53}{3.65}} \qquad ( Equation 3 )$$

It can be understood that the total fatty acid concentration obtained from the CADF is well in accord with that obtained from the GC measurement.

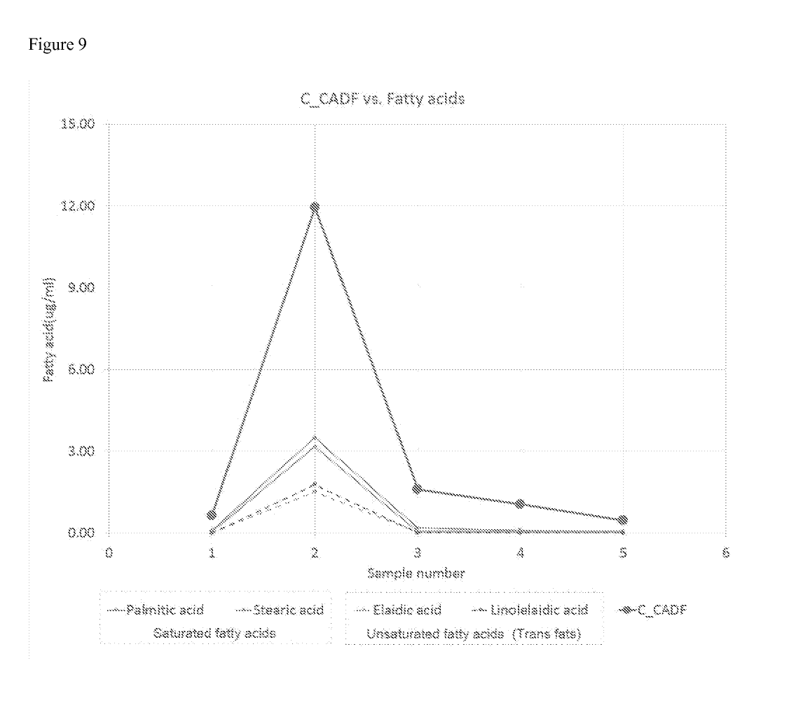

FIG. 9 is a graph showing the four important fatty acid concentrations and the total fatty acid concentration calculated from the CADF. The solid lines (blue and red) in FIG. 9 represent two fatty acids and the dotted lines (blue and grey) represent two unsaturated trans fatty acids.

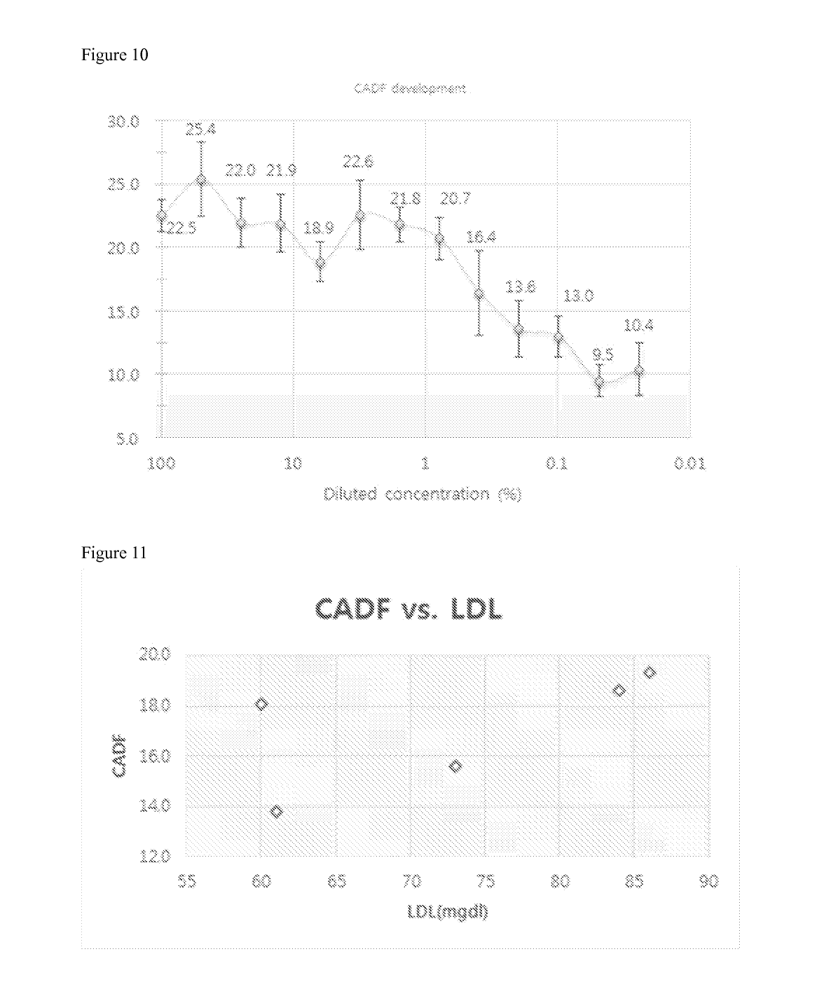

FIG. 10 is a graph showing the change of the CADF with diluting the blood plasma samples. As in the case of the urine, the CADF is proportional to the logarithm of the concentration.

FIG. 11 is a graph showing the LDL (low density lipoprotein) values obtained from the hyperlipidemia test results for the blood samples and the CADF values measured for the same blood samples. It can be understood from FIG. 11 that the LDL value is proportional to the CADF value. In addition, it revealed that the TG (triglycerides) or HDL (high density lipoprotein) value does not have any correlation with the CADF value. Saturated fatty acids or trans fatty acids raise levels of the LDL. Since the CADF is very sensitive to these harmful fatty acids, it can be understood that the CADF value is directly corresponding to the LDL value. Therefore, it is possible to measure the LDL value through measuring the CADF of blood. In addition, the CADF may be used as an indicator for preventing or managing cardiovascular diseases.

Based on the above, it can be understood by a person skilled in the art that the technical effects of the present invention are as follows:

Firstly, the present invention makes it possible to diagnose and prevent FES by monitoring the fat contents of blood and urine before, during, and after liposuction. Thus, measures may be taken to a patient before onset of FES.

Secondly, the present invention makes it possible to diagnose and prevent FES by monitoring the fat contents of blood and urine before, during, and after orthopedic surgery (operation). Thus, measures may be taken to a patient before onset of FES.

Thirdly, the method for measuring the CADF of the present invention may be applied to analytical techniques, for example, measuring the amount of fats contained in a sample. It is very difficult to quantitatively analyze fats through analytical techniques based on capillary columns since it is very hard to attach fluorescent substances to fats and fats do not absorb UV rays. In order to complement the conventional analytical techniques, the method for measuring the CADF of the present invention may be combined with the conventional analytical techniques. For example, the CADF according to the present invention can be applied to analyses of fats which cannot be measured by UV or fluorescence spectrometry.

Fourthly, the CADF according to the present invention can be applied to customized management of body fat in the field of health care/diet/obesity care, through monitoring of the CADF of body fluids such as blood, urine, etc.

Whilst some particular embodiments have been illustrated and described, it will be appreciated by persons skilled in the art that the present invention is not limited by what has been particularly shown by the exemplary embodiments described hereinabove. Thus, it should be understood that numerous additional embodiments are within the scope of the invention, mutatis mutandis.

What we claim is:

1. A method for measuring fat contents in a liquid based on a contact area diffusion factor, comprising:
placing liquid on a water-repellant surface as a droplet;
obtaining a magnified image from an image of said droplet;
obtaining a contact area (A(0)) between said repellant surface and said droplet from said magnified image; and
obtaining a contact area, A(t), between said repellant surface and said droplet after the lapse of predetermined time (t) from said magnified image, wherein said fat is determined to be present in said body fluid according to whether the value of A(t)−A(0) is positive or negative.

2. The method of claim 1, wherein said liquid is a body fluid.

3. The method of claim 2, wherein said body fluid is selected from the group consisting of serum, plasma, sweat and urine.
